(19) United States
(12) Patent Application Publication
(10) Pub. No.: US 2007/0014726 A1
(43) Pub. Date: Jan. 18, 2007

(54) Oxidation-resistant indicator macromolecule

(75) Inventors: Kandace Marie Merical, Mt. Airy, MD (US); Arthur E. Colvin JR., Mt. Airy, MD (US); Mark Alan Mortellaro, Frederick, MD (US)

Correspondence Address: ROTHWELL, FIGG, ERNST & MANBECK, P.C., 1425 K STREET, N.W., SUITE 800, WASHINGTON, DC, 20005, US

(73) Assignee: Sensors for Medicine and Science, Germantown, MD (US)

(21) Appl. No.: 11/487,435

(22) Filed: Jul. 17, 2006

Related U.S. Application Data
(60) Provisional application No. 60/699,844, filed on Jul. 18, 2005.

(51) Int. Cl. A61K 49/00

(52) U.S. Cl. 424/9.1

(57) ABSTRACT

In one aspect, the present invention relates to an implantable device for detecting the presence or concentration of an analyte in an aqueous environment in vivo. The device includes a macromolecule that comprises a copolymer of: a) one or more indicator component monomers which individually are not sufficiently water soluble to permit their use in an aqueous environment for detecting the presence or concentration of said analyte; b) one or more hydrophilic monomers; and c) one or more catalytic antioxidant monomers; such that the macromolecule is capable of detecting the presence or concentration of the analyte in an aqueous environment. The presence of the catalytic antioxidant reduces or prevents oxidative damage to the macromolecule.

### CROSS-REFERENCES TO RELATED APPLICATIONS

The present application claims the benefit of U.S. application Ser. No. 60/699,844 filed Jul. 18, 2005.

### BACKGROUND OF THE INVENTION

1. Field of the Invention

The present invention relates to an oxidation-resistant indicator macromolecule. Particularly, the present invention relates to a superoxide-resistant fluorescent indicator polymer macromolecule that has improved resistance to reactive oxygen species, such as superoxides, peroxy radicals, etc.

2. Description of the Related Art

Non-intrusive real-time monitoring of various human body conditions is very important and useful in the treatment of various diseases. Specifically, non-intrusive real-time monitoring of blood glucose levels is critical in the treatment of diabetics. Various methods have been developed and tried. Recently, a new technology was developed, which involves measuring the change of fluorescence of an indicator macromolecule (such as anthracene boronic acid derivatives) as a result of changes in the blood glucose levels with a detector incorporating the indicator macromolecule. This technology is described in, e.g., U.S. Pat. Nos. 6,304,766; 6,330,464; 6,344,360; 6,400,974; 6,711,423; 6,794,195 and 6,800,451, which are incorporated herein by reference in their entirety.

It has now been discovered by the present inventors that the fluorescence of such indicator macromolecules may diminish over time after implantation in animal bodies. This loss of fluorescence signal is attributed to oxidation by reactive oxygen species (“ROS”), including superoxide, peroxide and other ROSs known to exist in vivo. It is believed that these ROS are generated from phagocytic cells during inflammation, which is usually caused by implantation, and oxidize the fluorescent component of the indicator macromolecule. Although these ROSs typically have a half-life of up to a few seconds, they diffuse very rapidly through porous polymeric macromolecules due to their small size and often completely inactivate a fluorescent indicator macromolecule of 100 micron thickness over a period of time. Therefore, there is a need to develop an indicator macromolecule that is resistant to oxidation damages caused by ROS including superoxide, and maintain its fluorescence in a ROS-rich environment.

### BRIEF SUMMARY OF THE INVENTION

In one aspect, the present invention relates to an implantable device for detecting the presence or concentration of an analyte in an aqueous environment in vivo, said device including a macromolecule that comprises a copolymer of:
a) one or more indicator component monomers which individually are not sufficiently water soluble to permit their use in an aqueous environment for detecting the presence or concentration of said analyte;
b) one or more hydrophilic monomers; and
c) one or more catalytic antioxidant monomers;
   such that the macromolecule is capable of detecting the presence or concentration of said analyte in an aqueous environment.

In another aspect, the present invention relates to a method for detecting the presence or concentration of an analyte in a sample having an aqueous environment in vivo, said method comprising:
a) exposing the sample to an implantable device that includes an indicator macromolecule, said macromolecule comprising a copolymer of:
   i) one or more indicator component monomers which individually are not sufficiently water soluble to permit their use in an aqueous environment for detecting the presence or concentration of said analyte;
   ii) one or more hydrophilic monomers; and
   iii) one or more catalytic antioxidant monomers;
      such that the resulting macromolecule is capable of detecting the presence or concentration of said analyte in an aqueous environment, and wherein the indicator macromolecule has a detectable quality that changes in a concentration-dependent manner when said macromolecule is exposed to said analyte; and
b) measuring any change in said detectable quality to thereby determine the presence or concentration of said analyte in said sample.

In another aspect, the present invention relates to an implantable device that is capable of exhibiting an excimer effect, said device including a macromolecule which comprises a copolymer of:
a) one or more excimer forming monomers, the molecules of which are capable of exhibiting an excimer effect when suitably oriented with respect to each other; and
b) one or more other monomers comprising a catalytic antioxidant; such that the resulting macromolecule exhibits said excimer effect.

In another aspect, the present invention relates to a method for detecting the presence or concentration of an analyte in a sample in vivo, said method comprising:
a) exposing the sample to an implantable device that includes an indicator macromolecule, said macromolecule comprising a copolymer of:
   i) one or more indicator component monomers, the molecules of which are capable of exhibiting an excimer effect when suitably oriented with respect to each other, and which are also capable of detecting the presence or concentration of an analyte; and
   ii) one or more other monomers, comprising a catalytic antioxidant;
      such that the resulting macromolecule exhibits said excimer effect, and wherein the indicator macromolecule has a detectable quality that changes in a concentration-dependent manner when said macromolecule is exposed to said analyte; and
b) measuring any change in said detectable quality to thereby determine the presence or concentration of said analyte in said sample.

In another aspect, the present invention relates to an implantable device for detecting the presence or concentration of an analyte in an aqueous environment in vivo, said device including a macromolecule that comprises a copolymer of:
a) one or more indicator component monomers which individually are not sufficiently water soluble to permit their use in an aqueous environment for detecting the presence or concentration of said analyte; and
b) one or more hydrophilic monomers;
   such that the macromolecule is capable of detecting the presence or concentration of said analyte in an aqueous environment, and wherein at least one catalytic antioxidant is attached to said macromolecule.

### BRIEF DESCRIPTION OF THE DRAWINGS

FIG. 1 illustrates the results of the experiment described in Example 2.

### DETAILED DESCRIPTION OF INVENTION

According to the present invention, a catalytic antioxidant is incorporated into an implantable indicator macromolecule to confer protection from oxidation by reactive oxygen species. Many such antioxidants are known, and include any substance that when present at low concentrations compared to those of an oxidizable substrate, significantly delays or prevents the oxidation of that substrate. This includes not only species such as ascorbic acid, tocopherol, uric acid, glutathione, and Salen-Manganese complexes (see, Baker, et al., Eukarion, Inc, Bedford, Mass., The Journal of Pharm. and Exp. Therapeutics, Vol. 284, No. 1, p 215), but also enzymatic systems (e.g., superoxide dismutase, catalase, glutathione peroxidase and proteins used to sequester metals capable of HO′ production (e.g., transferrin, ferritin, ceruloplasmin, hemopexin, haptoglobulin, and albumin).

In a preferred embodiment, the catalytic antioxidant is a superoxide dismutase mimic. Superoxide dismutase (“SOD”) is a naturally occurring enzyme that catalyzes the dismutation of highly reactive superoxide to less reactive hydrogen peroxide and oxygen. As SOD is a protein, it is vulnerable to protease attack. It is also relatively large to be incorporated into a polymer structure and could be recognized by the host immune system as foreign protein matter. For example, commercially available SOD is normally isolated from bovine erythrocytes and is a homodimer having a molecular weight of about 32,500. On the other hand, several organic molecules have been developed to mimic SOD's superoxide dismutating activity. They are called superoxide dismutase mimics (“SODm”), and are disclosed for example in U.S. Pat. Nos. 6,214,817; 6,180,620, U.S. Pre-grant Publication 2004/0116332 and Udipi et al. (J. Biomed Mater Res, 51, 549-60 (2000)), which are incorporated herein by reference in their entirety.

In the present invention, catalytic antioxidants are incorporated in the indicator macromolecule to provide the macromolecule with resistance to oxidative damage caused by ROS, including superoxide. Preferably, the catalytic antioxidants are incorporated by copolymerization of catalytic antioxidant monomers, indicator component monomers and hydrophilic monomers. Catalytic antioxidants are redesigned as polymerizable catalytic antioxidant monomers by covalently attaching a catalytic antioxidant compound to a polymerizable monomer unit. Preferably, the polymerizable monomer unit is hydrophilic, but that is not always necessary. An example of such a redesign is shown below. In a preferred embodiment, the oxidation-resistant indicator macromolecule of the present invention comprises a copolymer of one or more indicator component monomers which individually are not sufficiently water soluble to permit their use in an aqueous environment for detecting the presence or concentration of said analyte; one or more hydrophilic monomers; such that the macromolecule is capable of detecting the presence or concentration of said analyte in an aqueous environment; and one or more catalytic antioxidant monomers; such that the macromolecule is capable of detecting the presence or concentration of said analyte in a ROS-challenged environment.

In a preferred embodiment, the catalytic antioxidant monomer comprises a superoxide dismutase mimic, which is a non-proteinaceous catalyst for the dismutation of superoxide. A superoxide dismutase mimic monomer is a superoxide dismutase mimic that has a reactive functional group that renders the monomer copolymerizable with at least one other monomer.

The copolymerization of antioxidant monomers with indicator component monomers provides the resulting indicator macromolecule maximum resistance to oxidative damage. Antioxidant moieties dispersed throughout the indicator macromolecule effectively degrade ROS that may diffuse into the indicator macromolecule. Immobilization of antioxidants within the indicator macromolecule by copolymerization minimizes the antioxidant's interference with normal healing process involving inflammation and ROSs. On the other hand, attaching antioxidants to the indicator macromolecule by chemical activation of antioxidant molecules and covalent attachment to the macromolecule is within the scope of the present invention, but is not preferred because such provides a limited protection only over the surface portion of the indicator macromolecule. Copolymerization of antioxidant monomers with indicator component monomers reduces manufacturing steps compared with attaching antioxidants to the indicator macromolecule by chemical activation of antioxidant molecules, and realizes production cost savings. Copolymerization allows more control over the ratio between antioxidant moieties and indicator moieties and higher concentration of antioxidant moieties in the indicator macromolecule than attaching antioxidants to the indicator macromolecule by chemical activation of antioxidant molecules.

It would be readily apparent how to convert an antioxidant to an antioxidant monomer. One such scheme is shown below, with respect to a SODm: 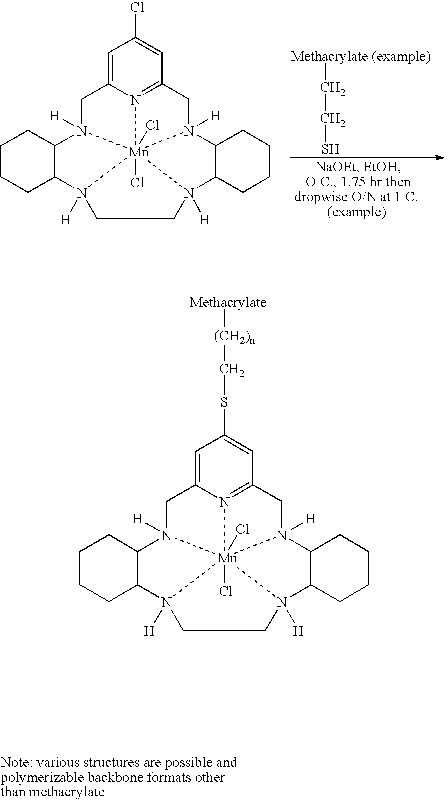
In addition to the SODm shown above, many other SODm's are known and are applicable to the present invention. Examples include the following:

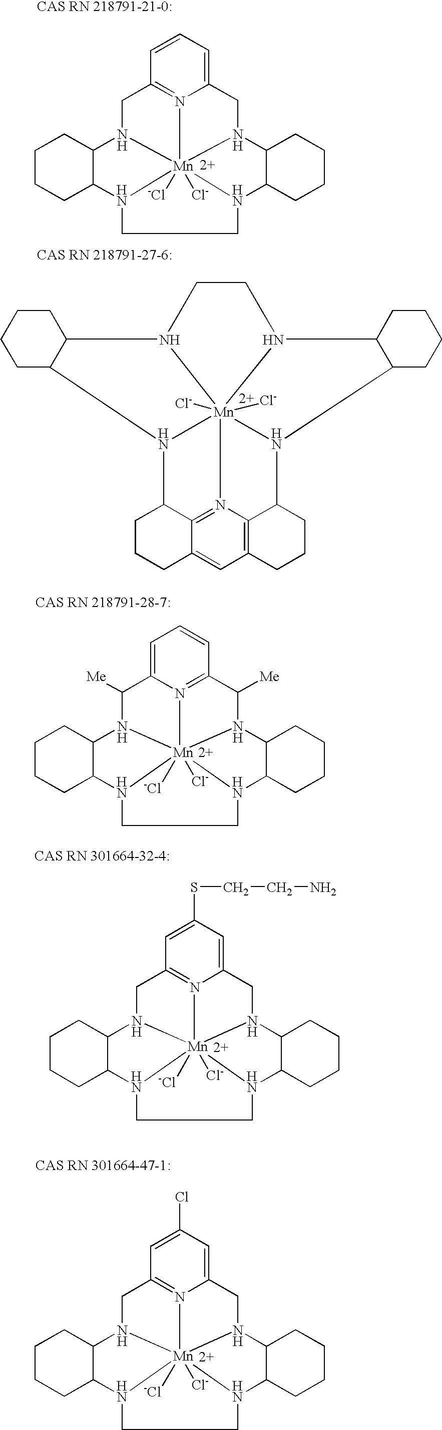

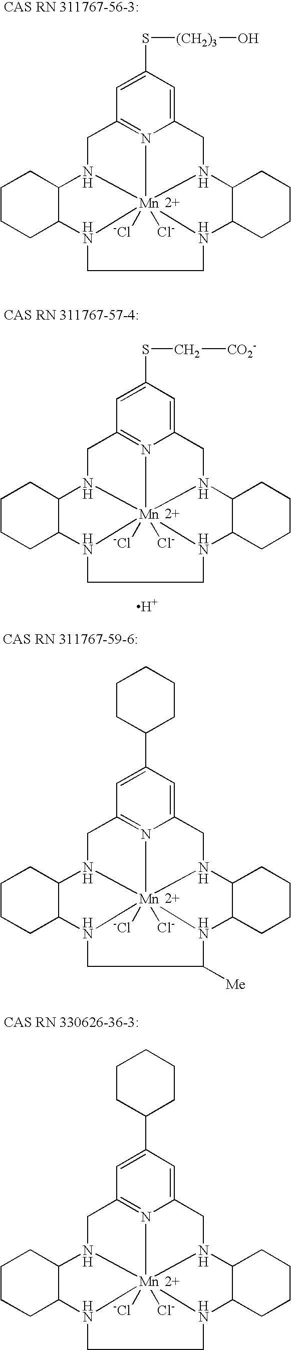

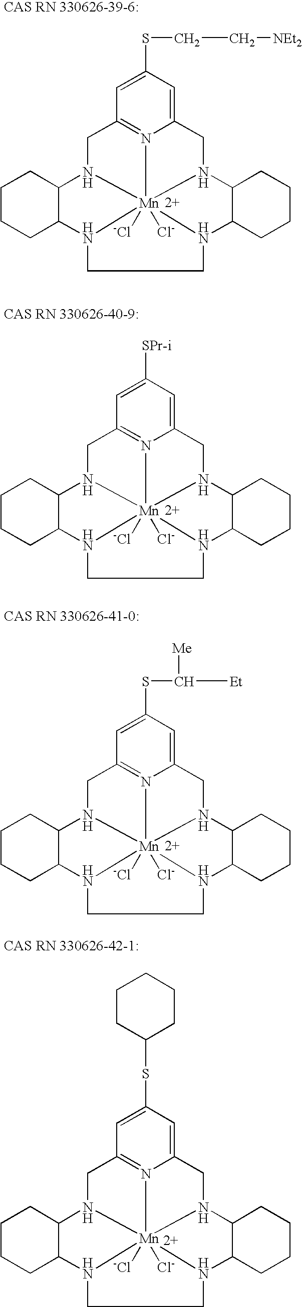

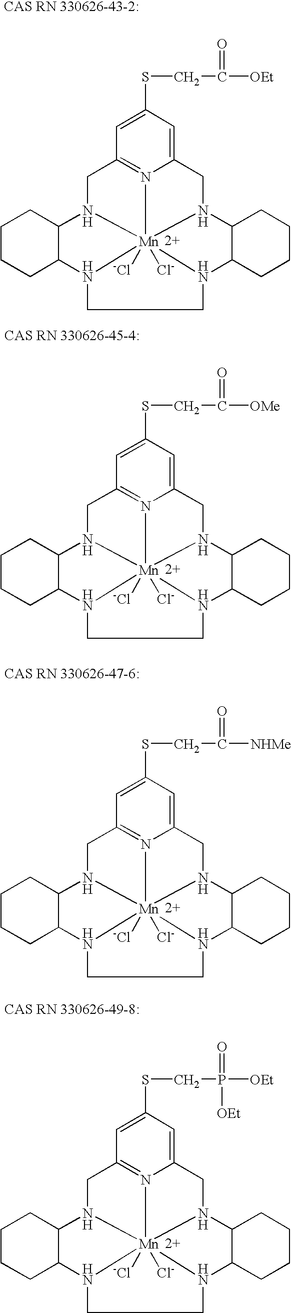

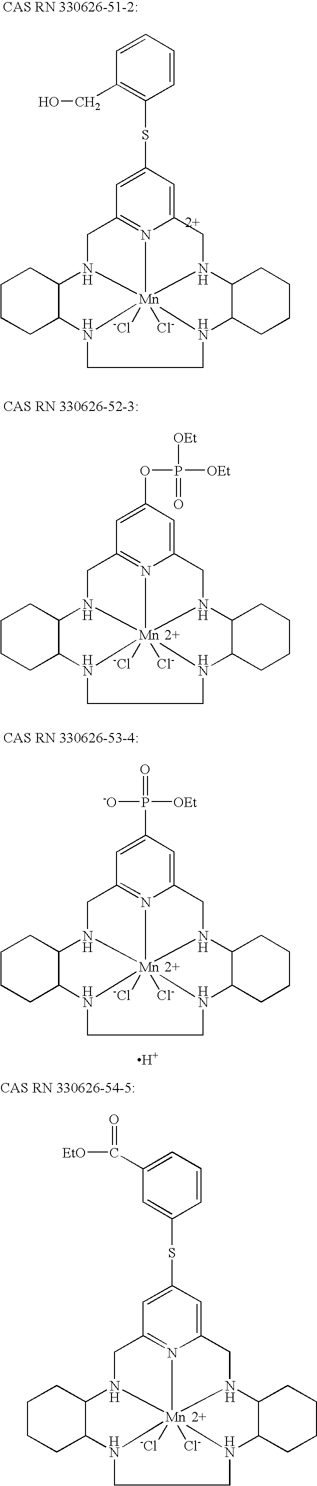

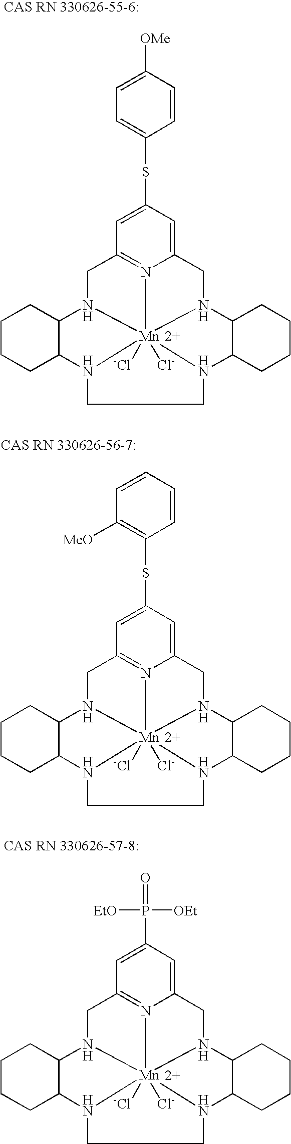

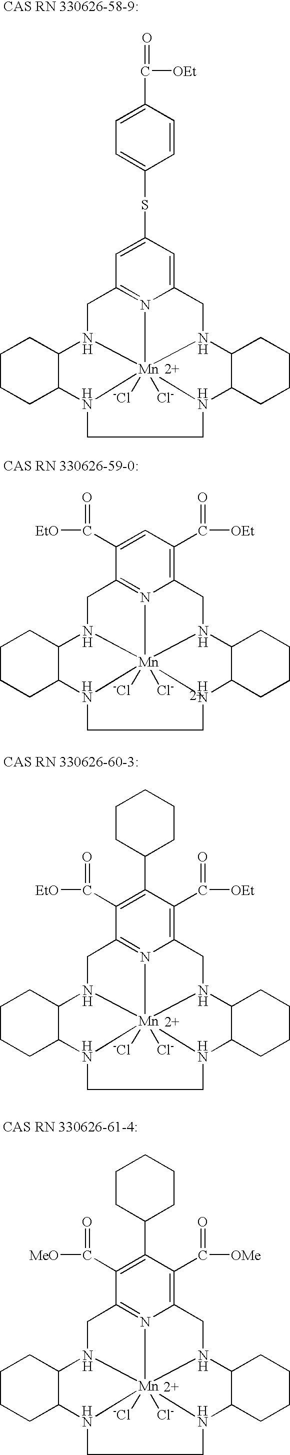

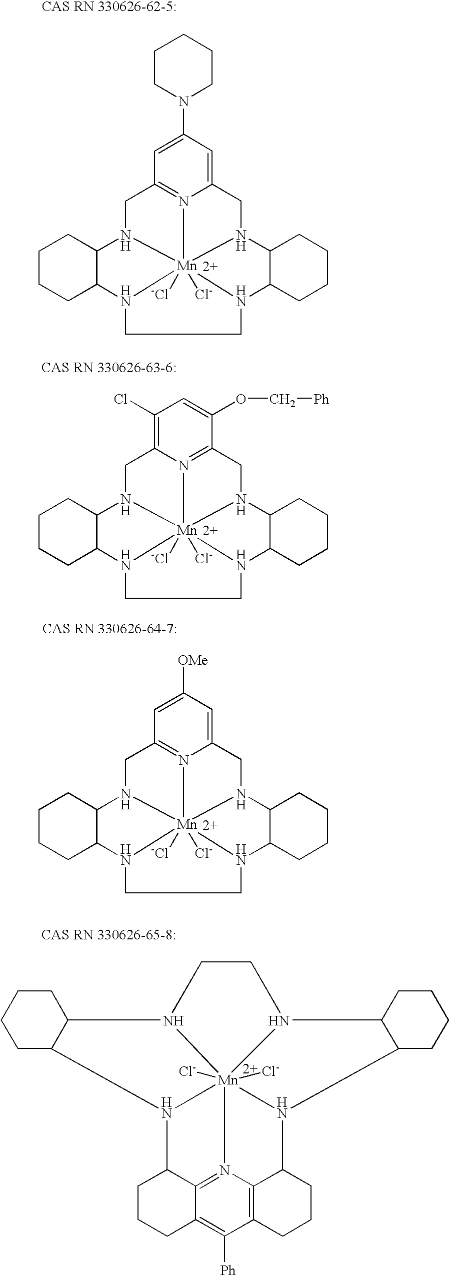

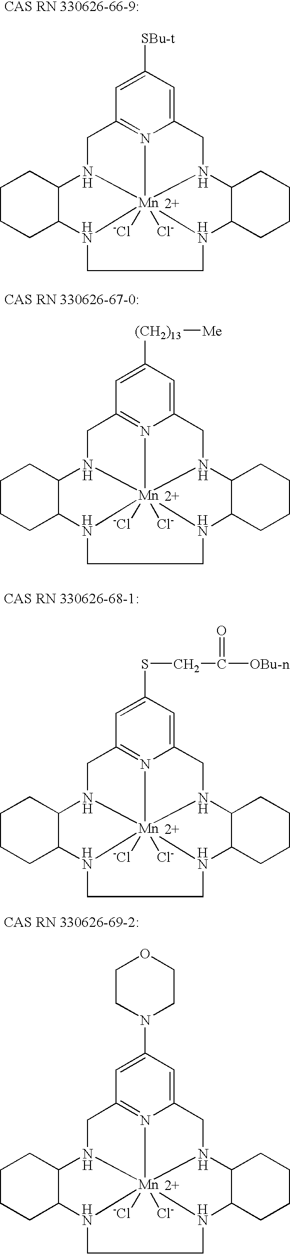

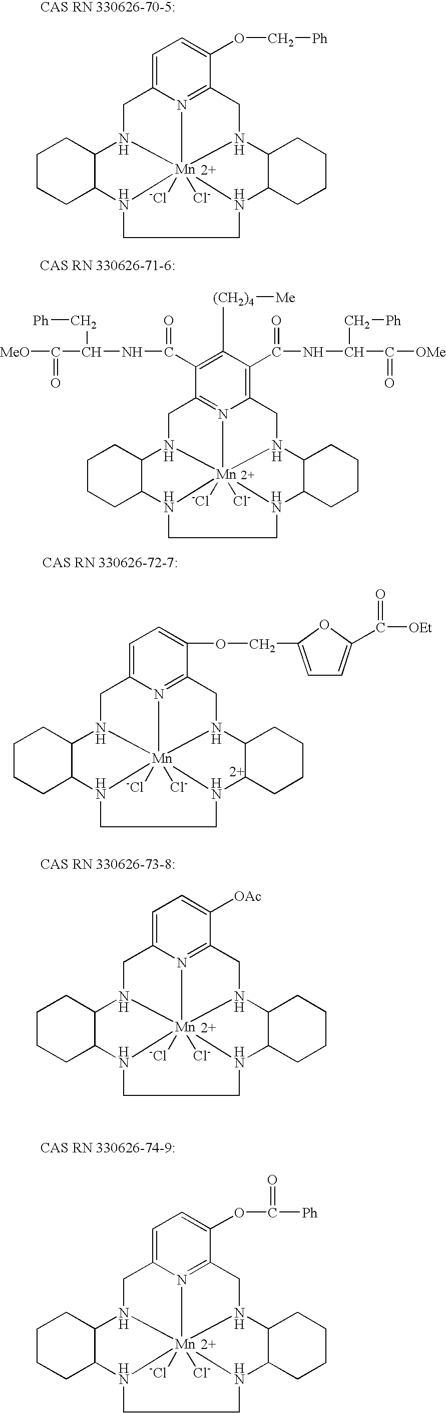

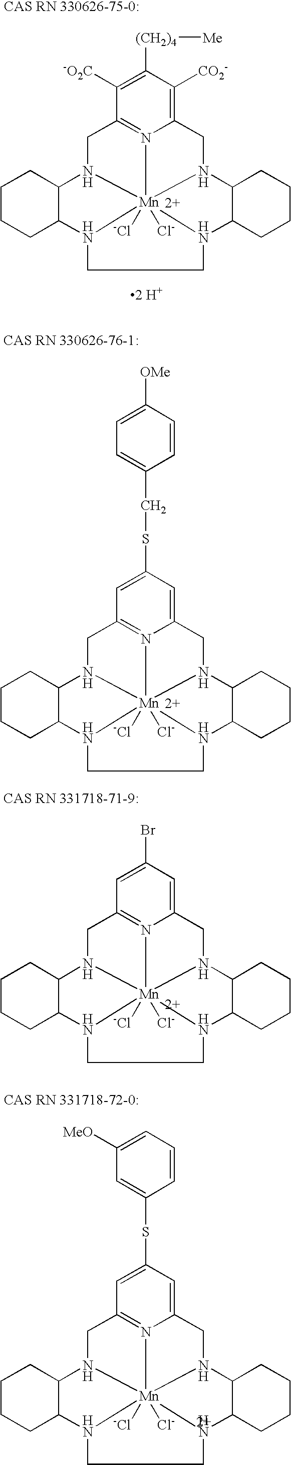

Suitable indicator components include indicator molecules which are insoluble or sparingly soluble in water, and whose analyte is at least sparingly soluble in water. Suitable analytes include glucose, fructose and other vicinal diols; α-hydroxy acids; β-keto acids; oxygen; carbon dioxide; various ions such as zinc, potassium, hydrogen (pH measurement), carbonate, toxins, minerals, hormones, etc. It will be appreciated that within the scope of indicator component monomer as used herein are included mixtures of two or more individual monomers (at least one of which is not sufficiently soluble to function adequately in an aqueous environment) which, when incorporated into the macromolecules of the present invention, function as an indicator.

Many such indicator components are known. For example, the compounds depicted in U.S. Pat. No. 5,503,770 are useful for detecting saccharides such as glucose, but are sparingly soluble to insoluble in water. Other classes of indicators include the lanthanide chelates disclosed in U.S. Pat. No. 6,344,360; polyaromatic hydrocarbons and their derivatives; the indicators disclosed in U.S. Pat. No. 6,800,451, which describes indicators having two recognition elements capable of discriminating between glucose and interfering α-hydroxy acids or β-diketones, etc.

The indicator components of the present invention will generally have a detectable quality that changes in a concentration-dependent manner when the macromolecule is exposed to the analyte to be measured. Many such qualities are known and may be used in the present invention. For example, the indicator may include a luminescent (fluorescent or phosphorescent) or chemiluminescent moiety, an absorbance based moiety, etc. The indicator may include an energy donor moiety and an energy acceptor moiety, each spaced such that there is a detectable change when the macromolecule interacts with the analyte. The indicator may include a fluorophore and a quencher, configured such that the fluorophore is quenched by the quencher when the analyte is absent. In that situation, when the analyte is present, the indicator undergoes a configurational change which causes the quencher to move sufficiently distant from the fluorophore so that fluorescence is emitted. Conversely, the fluorophore and quencher may be configured such that in the absence of analyte, they are sufficiently separated and the fluorophore emits fluorescence; upon interaction with the analyte, the fluorophore and quencher are moved in sufficient proximity to cause quenching. The configurational change concept is described in more detail in published U.S. application US 2002/0094586, incorporated herein by reference.

Other detectable moieties include those whose fluorescence is affected by analyte interaction via photoinduced electron transfer or inductive effects. These include the lanthanide chelates disclosed in U.S. Pat. No. 6,344,360; polyaromatic hydrocarbons and their derivatives; coumarins; BODIPY® (Molecular Probes, Eugene, Oreg.); dansyl; catechols; etc. Another class of moieties include those whose absorbance spectrum changes upon interaction of the indicator compound with the analyte, including Alizarin Red, etc. Another class of moieties include those whose fluorescence is modulated by proximity effects, e.g., energy donor/acceptor pairs such as dansyl/dabsyl, etc.

Preferably, the detectable quality is a detectable optical or spectral change, such as changes in absorptive characteristics (e.g., absorptivity and/or spectral shift), in fluorescent decay time (determined by time domain or frequency domain measurement), fluorescent intensity, fluorescent anisotropy or polarization; a spectral shift of the emission spectrum; a change in time-resolved anisotropy decay (determined by time domain or frequency domain measurement), etc.

Suitable hydrophilic monomers should be sufficiently hydrophilic so as to overcome the sum of the hydrophobic indicator component monomers, such that the resultant indicator macromolecule is capable of functioning in an aqueous environment. It will be readily apparent that a wide variety of hydrophilic monomers are suitable for use in the present invention. For example, suitable hydrophilic monomers include methacrylamides, methacrylates, methacrylic acid, acrylic acid, dimethylacrylamide, TMAMA, vinyls, polysaccharides, polyamides, polyamino acids, hydrophilic silanes or siloxanes, etc., as well as mixtures of two or more different monomers.

Suitable hydrophilic monomers for a given application will vary according to a number of factors, including intended temperature of operation, salinity, pH, presence and identity of other solutes, ionic strength, etc. It would be readily apparent that the degree of hydrophilicity of the hydrophilic monomer or the indicator macromolecule can be increased by adding additional functional constituents such as ions (e.g., sulfonate, quartenary amine, carboxyl, etc.), polar moieties (e.g., hydroxyl, sulfhydryl, amines, carbonyl, amides, etc.), halogens, etc.

It will be appreciated that the molar ratios of the monomers used herein may vary widely depending on the specific application desired. Preferred ratios of hydroplilic monomer:indicator component monomer range from about 2:1 to about 1000:1, more preferably from about 5:1 to about 50:1.

The indicator macromolecules of the present invention may generally be synthesized by simply copolymerizing at least one indicator component monomer, with at least one hydrophilic monomer and with the antioxidant monomer. Optimum polymerization conditions (time, temperature, catalyst, etc.) will vary according to the specific reactants and the application of the final product, and can easily be established by one of ordinary skill.

It will be appreciated that the indicator macromolecules of the present invention may have any desired extent of water solubility. For example, the indicator macromolecule of Examples 1 and 2 of U.S. Pat. No. 6,794,195 is very soluble, readily dissolving in aqueous solution. On the other hand, indicator macromolecules containing, for example, the hydrophilic monomer HEMA (hydroxyethyl methacrylate) or other common hydrogel constituents, can be non-soluble yet hydrophilic.

The soluble indicator macromolecules may be used directly in solution if so desired. On the other hand, if the desired application so requires, the indicator macromolecule may be immobilized (such as by mechanical entrapment, covalent or ionic attachment or other means) onto or within an insoluble surface or matrix such as glass, plastic, polymeric materials, etc. When the indicator macromolecule is entrapped within, for example, another polymer, the entrapping material preferably should be sufficiently permeable to the analyte to allow suitable interaction between the analyte and the indicator components in the macromolecule.

Many uses exist for the indicator macromolecules of the present invention. For example, the indicator macromolecules can be used as indicator molecules for detecting sub-levels or supra-levels of glucose in blood, tissues, urine, etc., thus providing valuable information for diagnosing or monitoring such diseases as diabetes and adrenal insufficiency.

When the indicator macromolecules incorporate fluorescent indicator substituents, various detection techniques also are known in the art that can make use of the macromolecules of the present invention. For example, the macromolecules of the invention can be used in fluorescent sensing devices (e.g., U.S. Pat. No. 5,517,313).

U.S. Pat. No. 5,517,313, the disclosure of which is incorporated herein by reference, describes a fluorescence sensing device in which the macromolecules of the present invention can be used to determine the presence or concentration of an analyte such as glucose or other vicinal diol compound in a liquid medium. The sensing device comprises a layered array of a fluorescent indicator molecule-containing matrix (hereafter “fluorescent matrix”), a high-pass filter and a photodetector. In this device, a light source, preferably a light-emitting diode (“LED”), is located at least partially within the indicator material, or in a waveguide upon which the indicator matrix is disposed, such that incident light from the light source causes the indicator molecules to fluoresce. The high-pass filter allows emitted light to reach the photodetector, while filtering out scattered incident light from the light source.

The fluorescence of the indicator molecules employed in the device described in U.S. Pat. No. 5,517,313 is modulated, e.g., attenuated or enhanced, by the local presence of an analyte such as glucose or other cis-diol compound.

In the sensor described in U.S. Pat. No. 5,517,313, the material which contains the indicator molecule is permeable to the analyte. Thus, the analyte can diffuse into the material from the surrounding test medium, thereby affecting the fluorescence emitted by the indicator molecules. The light source, indicator molecule-containing material, high-pass filter and photodetector are configured such that at least a portion of the fluorescence emitted by the indicator molecules impacts the photodetector, generating an electrical signal which is indicative of the concentration of the analyte (e.g., glucose) in the surrounding medium.

In accordance with other possible embodiments for using the indicator macromolecules of the present invention, sensing devices also are described in U.S. Pat. Nos. 5,910,661, 5,917,605 and 5,894,351, all incorporated herein by reference.

The macromolecules of the present invention may be used in an implantable device, for example to continuously monitor an analyte in vivo (such as blood or tissue glucose levels). Suitable devices are described in, for example, U.S. Pat. Nos. 6,330,464, 5,833,603, 6,002,954 and 6,011,984, all incorporated herein by reference.

The macromolecules of the present invention have unique advantages. For example, absorbance of a sample is directly proportional to both the concentration of the absorber and the sample path length. Thus, in an absorbance-based assay, it is apparent that for a given level of absorbance, the sample path length may be greatly reduced if the absorber concentration is greatly increased. That desirable increase in concentration may be accomplished by decreasing the ratio of the hydrophilic monomer:indicator component monomer. In effect, the present invention allows the localized concentration of much more absorber component into a limited space, thereby increasing the absorbance per unit thickness. Thus the present invention additionally allows use of much smaller equipment when performing absorbance-based assays. It will also be apparent that for any optically-based assay, including fluorescence based assays, the ability to greatly increase the local concentration of the indicator component offers several advantages. For example, a higher local concentration of the indicator component can permit the utilization of thinner layers of indicator macromolecule, which in turn can greatly reduce the response time of the macromolecule to the presence or concentration of the analyte. Further, it can result in a higher extinction of excitation light, which can desirably reduce the incidence of autofluorescence when working in tissue systems or physiological solutions. For example, when working with a fluorescence based macromolecule, non-absorbed excitation light can interact with, e.g., NADH, tryptophan, tyrosine, etc. which may be present in tissue or physiological solutions resulting in undesirable interfering fluorescent emission from those moieties. Having a high local concentration of indicator component with high absorption can reduce that undesired interfering emission. Additionally, when utilizing an absorbance-based macromolecule in tissue or physiological solutions, it is desirable to reduce the amount of the source radiation that is reflected in potentially varying amounts by components in surrounding tissue or fluid, such as bilirubin, e.g. Therefore, having a high local concentration of indicator component with high absorption can reduce that undesired effect.

The present invention may also be used in an excimer-forming system as described in U.S. Pat. No. 6,794,195. By way of background, when two planar molecules with aromatic structure (such as is common with fluorophores) are concentrated to a point where their pi electron orbital lobes may overlap, a resonance condition can then occur for some species where the resonance from overlap results in a hybrid (couplet) structure which is energy favorable and stable. These two planar molecules become oriented in a coplanar configuration like two slices of bread on a sandwich with their electron clouds overlapping between them. For fluorescent planar species, emission occurs at wavelengths of substantially lower energy than for the parent species. Molecules able to form such favorable resonant configurations are known as excimers. As used herein, an excimer effect refers to the resulting characteristic longer wavelength emission from excimers.

Some examples of typical excimer-forming polyaromatic hydrocarbons include anthracene and pyrene. There are many others. An example is the anthracene derivative (boronate included), the indicator component used in Examples 1 and 2 of U.S. Pat. No. 6,794,195. Although anthracene is known to form excimers in solution, one must be able to concentrate the molecule to sufficiently high levels to observe any excimer character. In the case of the anthracene derivative of Examples 1 and 2, the molecule is insoluble in water and insufficiently soluble in a solvent such as methanol to observe excimer characteristics. In U.S. Pat. No. 6,794,195, the relative concentration of the anthracene derivative monomer was increased in proportion to the hydrophilic monomer in the copolymer from 500:1, 400:1, 200:1, 100:1, 50:1, 25:1, 15:1 and then 5:1. All had the characteristic blue emission at 417 nm of the anthracene derivative except at 5:1 ratio, a green emission suddenly appears. This green emission is that of an excimer hybrid and the emission has been shifted downfield by approximately 100+nanometers (˜515-570 nm, green). The concentration of the overall solution does not need to be high since the distance between planar species is being controlled by placement along the polymer backbone rather than soluble concentration in 3-D space.

As noted in U.S. Pat. No. 6,794,195 the excimer emission region is not responsive to changes in analyte concentration, but is responsive to all other aspects of the system analyzed, such as excitation intensity, temperature, and pH. As a result, it is believed that the present indicator macromolecules incorporating a SODm may serve as both an indicator and an internal reference. For example, an ideal referencing scheme is one where the emission intensity at an indicator wavelength (i.e., the wavelength influenced by the analyte) is divided optically using select bandpass filters, by the emission intensity at the excimer wavelength. The resultant value corrects for interfering factors which affect fluorescent emission properties, such as fluorescent quenching by, e.g., oxygen, drift and error in pH, power factors and drift affecting LED intensity, ambient temperature excursions, etc.

It will be readily appreciated that the macromolecules of the present invention which exhibit an excimer effect will be useful in both aqueous and non-aqueous environments. Consequently, those macromolecules, as well as the component monomers (excimer-forming and other monomer), may range from hydrophilic to hydrophobic, depending upon the desired application.

Also, when the excimer macromolecules of the present invention are used to detect the presence or concentration of an analyte, the macromolecule may be used directly in solution, or may be immobilized as described above.

The macromolecules of the present invention can be prepared by persons skilled in the art without an undue amount of experimentation using readily known reaction mechanisms and reagents.

Suitable preferred indicator component monomers include:
9,10-bis[N-(2-boronobenzyl)-N-[2-(2-methacroyloxyethoxy)ethylamino]methyl]anthracene;
9,10-bis[N-(2-boronobenzyl)-N-[3-(methacrylamido)-propylamino]methylanthracene;
9,10-bis[N-[2-(5,5-dimethyl-[1,3,2]dioxaborinan-2-yl)benzyl]-N-[2-(2-methacroyloxyethoxy)ethylamino]methyl]anthracene;
9,10-bis[N-[2-(5,5-dimethyl-[1,3,2]dioxaborinan-2-yl)benzyl]-N-[3-(methacrylamido)propylamino]methyl]anthracene;
9-[[N-methacryloylaminopropyl-N-(o-boronobenzyl)amino]-methyl]anthracene;
9-[N-(2-boronobenzyl)-N-[2-(2-methacroyloxyethoxy)-ethylamino]methyl]-10-[N-[2-boronobenzyl)]-N-[2-(2-hydroxyethoxy)ethylamino]methyl]anthracene;
9-[N-[2-(5,5-dimethyl-[1,3,2]dioxaborinan-2-yl)benzyl]-N-[2-(2-methacroyloxyethoxy)ethylamino]methyl]-10-[N-[2-(5,5-dimethyl-[1,3,2]dioxaborinan-2-yl)benzyl]-N-[2-(2-hydroxyethoxy)-ethylamino]methyl]anthracene;
α,α′-bis[N-[2-(5,5-dimethyl-[1,3,2]dioxaborinan-2-yl)benzyl]-N-[3-(methacrylamido)propylamino]-1,4-xylene;
9-[N-(2-boronobenzyl)-N-[3-(methacrylamido)propylamino]methyl]-10-[N-(2-boronobenzyl)-N-[2-(carboxyethyl)amino]methyl]anthracene;
9-[N-(2-boronobenzyl)-N-[3-(methacrylamido)propylamino]methyl]-10-[N-(2-boronobenzyl)-N-[6-(cyclohexanecarboxamido)hexylamino]methyl]anthracene; and
9-[N-[2-(4,4,5,5-tetramethyl-1,3,2-dioxaborolano)benzyl]-N-[3-(methacrylamido)propylamino]methyl]-10-[N—[2-(4,4,5,5-tetramethyl-1,3,2-dioxaborolano)benzyl]-N-[2-(carboxyethyl)amino]methyl]anthracene.

Incorporation of the catalytic antioxidants into implantable sensors is believed to confer two direct protections to the device. One protects the indicator, the other protects the overall polymeric material properties.

Chemical analysis of explanted, attacked sensors shows that the exposure to ROS results in the indicator system being oxidized, resulting in reduced modulation with glucose.

Analysis of explanted, attacked sensors also shows the direct effect of radicals on the material properties of the sensor. When the graft and sensor material is polymeric consisting of a HEMA copolymer in the graft region and a PMMA encasement beneath, both materials are exposed to the ambient implant (in-vivo) environment. These reactive oxygen species also cause unwanted additional crosslinking of existing polymers. Additional crosslinking causes an increased density of the polymeric matrix resulting in a profound change of material mechanical properties (as is typical with increased crosslinking by any method). The increased crosslinking from ROS causes the exposed polymers of the sensor construct to become denser and more brittle, and therefore more susceptible to brittle fracture. The increased crosslinking within the macromolecular indicator matrix also alters the optical and diffusion properties within the porous matrix by altering the pore size and porosity. The catalytic antioxidant incorporated into a material can protect both the mechanical properties of the material as well as protect against signal loss of fluorescence.

### EXAMPLE 1

The following working example utilizes the following synthesis scheme:

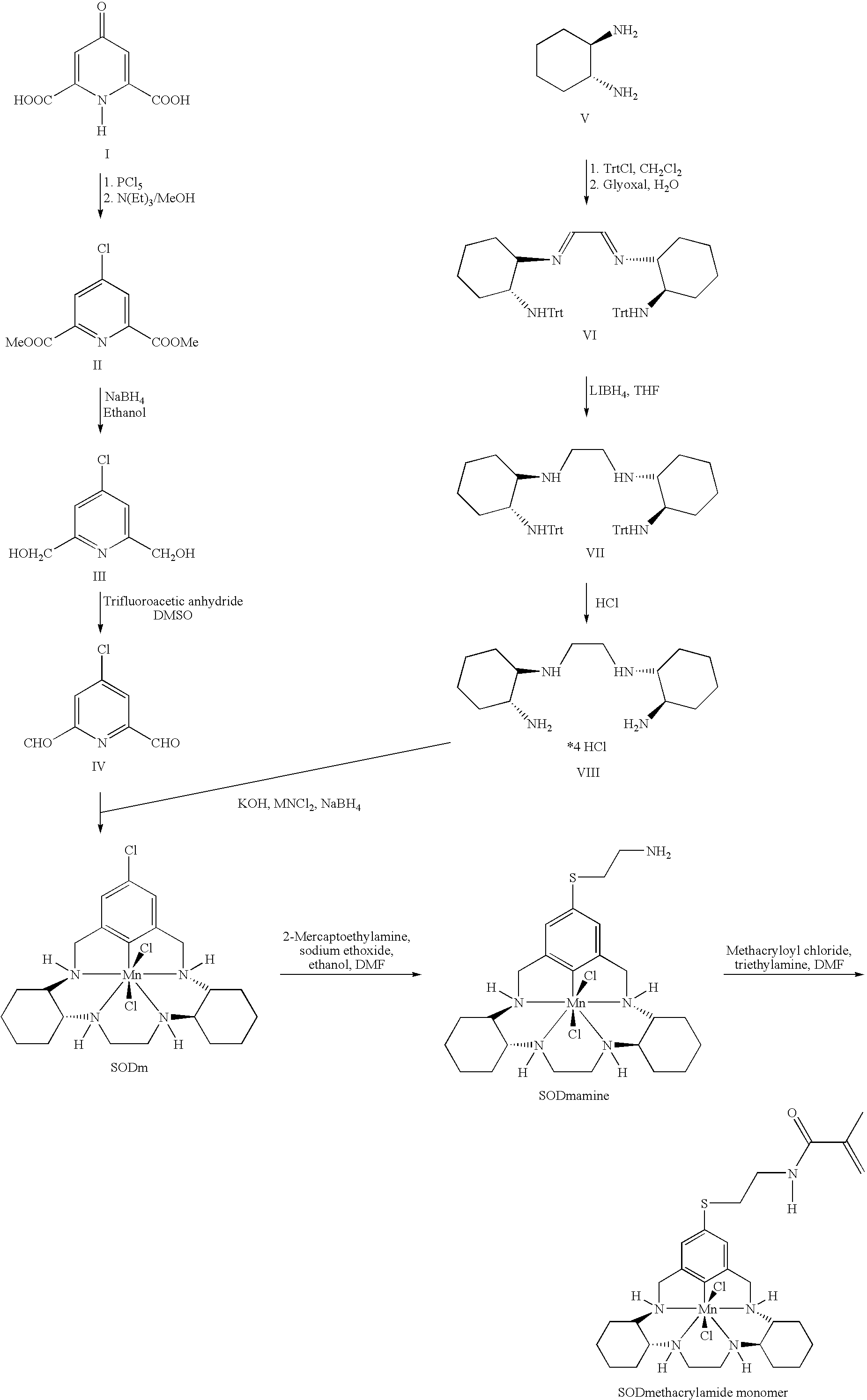

The synthesis of the SODmethacrylamide monomer began with commercially available compounds I and V. The syntheses of intermediates II, VI, VII and VIII were accomplished according to the methods described in U.S. Pat. No. 6,214,817 B 1, incorporated herein by reference. The other intermediates were prepared as described below.

### Synthesis of Intermediate III

Compound II (29.4 g, 128 mmol) was suspended in anhydrous ethanol (800 mL) and cooled to 0° C. To this suspension, sodium borohydride (9.66 g, 256 mmol) was added in small portions, the mixture stirred at 0° C. for one hour then allowed to warm to room temperature. Stirring was continued for 3 hours, then the mixture was heated to reflux overnight. The reaction mixture was cooled to room temperature, acetone (100 mL) was added and the solution heated to reflux for one hour. The reaction mixture was concentrated under vacuum to remove ethanol and acetone. The resulting white solid was suspended in ethyl acetate (600 mL) and saturated sodium bicarbonate solution (150 mL). The two phase mixture was stirred at room temperature for 30 minutes. The organic layer was separated and the solvent removed under vacuum to yield compound III as a white powder (14 g, 63%): ¹H NMR (CD₃OD, 400 MHz) δ 7.43 (s, 2H), 4.85 (s, 2H), 4.65 (s, 4H).

### Synthesis of Intermediate IV

Anhydrous DMSO (18.06 mL, 254.3 mmol) was mixed with anhydrous dichloromethane (126 mL) then cooled to −60° C. with a dry ice/acetone bath. Trifluoroacetic anhydride (32 mL, 230 mmol) was added dropwise to the DMSO reaction mixture at −60° C. and under Argon. After 2 hours, a solution of III (14 g, 80.7 mmol) in DMSO (47 mL) was added and the reaction solution maintained at −40° C. to −50° C. for an additional 2 hours. Triethylamine (59.1 mL, 424 mmol) was then added. After 30 minutes, the reaction mixture was slowly warmed to room temperature and then stirred an additional 2 hours. Hydrochloric acid (2 M, 60 mL) was added, stirred for 20 minutes then water (350 mL) and dichloromethane (400 mL) were added. The layers were separated and the aqueous layer extracted with more dichloromethane (2×400 mL). The combined organic solutions were washed with saturated NaCl solution, dried over sodium sulfate then the solvent was removed under vacuum. The crude yellow solid was purified by flash chromatography on silica gel eluted with dichloromethane followed by 2% methanol in dichloromethane. Compound IV was isolated as a yellow powder (9.78 g, 71%). ¹H NMR (CDCl₃, 400 MHz) δ 10.1 (s, 2 H), 8.2 (s, 2 H).

### Synthesis of SODm

Compound VIII (3.19 g, 7.98 mmol) was suspended in absolute ethanol (80 mL) and stirred under Argon for 10 minutes. Powdered KOH (2.08 g, 31.5 mmol) was added in two equal portions at 30 minute intervals and stirring continued for 1 hour. MnCl₂ (1.05 g, 8.33 mmol) was added and the suspension stirred for another hour. Compound IV (1.34 g, 7.88 mmol) was then added and the solution stirred under Argon overnight. The orange suspension was then heated to 60° C. for a second night at which point TLC analysis showed no starting material and a single new spot. The solution was allowed to cool to room temperature, methanol (3.5 mL) was added and the solution cooled to 0° C. NaBH₄ (0.67 g, 17.7 mmol) was added in small portions, the suspension was stirred at 0° C. for 1 hour then was allowed to come to room temperature. HPLC analysis showed only 80% reduction at which point more NaBH₄ (60 mg, 1.6 mmol) was added and stirring continued for 1 hour. Water (10 mL) was added to quench the reaction and the solvents were removed under vacuum. The residue was dissolved in water (75 mL), extracted with dichloromethane (4×75 mL) and the combined organic solutions were removed under vacuum. The crude material was purified by flash chromatography on silica gel 60 eluted with chloroform followed by 1-3% methanol in chloroform. SODm (3.17 g, 78%) was isolated as an off-white powder. ESI MS mz/481 (M-Cl)⁺, 392 (M-Cl—HCl)⁺, 223 (M-2 Cl)²⁺.

### Synthesis of SODmamine

2-Mercaptoethanol (0.608 g, 7.88 mmol) from a freshly opened bottle was dissolved in anhydrous, Argon sparged ethanol (40 mL) in an oven dried flask and then capped with an Argon balloon. The solution was cooled in an ice water bath then ethanolic sodium ethoxide (3.2 mL, 8.58 mmol, 1.1 eq) was added dropwise by pipette. Stirring was continued in the ice bath for 40 minutes then at room temperature for 4.5 hours while under an Argon stream. This solution was added dropwise (via addition funnel) into a solution of SODm (2.03 g, 3.93 mmol) in anhydrous DMF (40 mL) at 0° C. The reaction mixture was allowed to stir overnight at room temperature under a stream of Argon. The solvent was removed under vacuum, and water (100 mL) was added to the residue and then extracted with chloroform (4×100 mL). The chloroform was removed under vacuum and the crude material was purified by flash chromatography on silica gel 60 eluted with dichloromethane and then 1-10% methanol in dichloromethane. SODmamine was isolated as a yellow powder (1.34 g, 61%). FAB MS: m/z 557 M⁺, 522 (M-Cl)⁺.

### Synthesis of SODmethacrylamide

SODmamine (1.19 g, 2.14 mmol) was weighed into an oven dried flask. Dichloromethane (120 mL) was added and the solution was stirred under Argon. Triethylamine (0.60 mL, 4.3 mmol, 2.0 eq) was added all at once. Methacryloyl chloride (340 microliters, 3.50 mmol, 1.6 eq) was then added dropwise by glass pipette over ˜3 minutes. The solution was then stirred at room temperature under an Argon balloon for 3 hours by which time HPLC analysis showed complete consumption of the starting material and the appearance of a new peak. The dichloromethane solution was washed with water (3×120 mL) and the solvent was removed under vacuum to yield a yellow solid (1.55 g). The crude material was purified by flash chromatography on silica gel 60 eluted with dichloromethane then 1-5% methanol in dichloromethane. SODmethacrylamide was isolated as a light yellow powder (0.917 g, 68% yield). FAB MS: m/z 625 M⁺, 590 (M-Cl)⁺.

### Preparation of HEMA Slab Gels with SODmethacrylamide

The SODmetharylamide Indicator monomer was incorporated into HEMA slab gels according to the following procedure.

Prep of HEA:

3000 μL Inhibitor free HEMA

120 μL Acrylic Acid, Aldrich (147230-100G, lot 21311HB)

6.2 μL EGDMA, Aldrich (335681-100, lot 10701DB)

Prep of 2% VAZO:

15.1 mg VAZO, WAKO (VA-044)

755 μL water, Fluka (95305)

Prep of Indicator in HEA:

7.0 mg carboxy indicator; 9-[N-[2-(4,4,5,5-tetramethyl-1,3,2-dioxaborolano)benzyl]-N-[3-(methacrylamido)propylamino]methyl]-10-[N—[2-(4,4,5,5-tetramethyl-1,3,2-dioxaborolano)benzyl]-N-[2-(carboxyethyl)amino]methyl]anthracene, preparation described in, e.g., Example 12 of U.S. Pat. No. 6,800,451, incorporated herein by reference

500 μL HEA (see above)

Prep of slabs with 1% w/v SODmethacrylamide

1.8 mg of SODmethacrylamide (lot #127-31A) synthesized in house

180 μL of Indicator in HEA (see above)

420 μL of Fluka water (#95305)

1) Mix the three reactants and sparge under N₂ for two minutes
2) Add 20.1 μL 2% VAZO (see above)
3) Fill 3 100 micron slab molds (the molds were constructed from a 100 micron shim sandwiched between two glass slides).
4) All slabs were polymerized at 60° C. and 100% RH for 18 hours. The resulting white gels were then washed in PBS overnight.
5) Lastly, white gels were soaked in a saturated Manganese Chloride solution in PBS overnight, and then placed into PBS solution for storage until use.

### EXAMPLE 2

### Cytochrome C Assay on Ground Slab Gels

The assay below is designed to test the polymeric material's properties to resist superoxide radicals and thus protect the indicator monomer component of the material. The reduction of cytochrome c to quantitate O₂⁻ production in solution is well established. (J. Biol. Chem., 1969, 244(22), 6049; Am. J. Respir. Crit. Care Med. 1997, 156, 140-145). Superoxide radicals reduce cytochrome c, resulting in an increase in absorbance at 550 nm:
cytochrome c (oxidized)+O₂⁻→cytochrome c (reduced)+O₂

Superoxide dismutase (SOD) or SOD mimics inhibit cytochrome c reduction by scavenging O₂⁻. That scavenging ability is quantitated as “% inhibition of cytochrome c” and is calculated by comparison to controls lacking in sod or sod mimics.

Xanthine with xanthine oxidase is used to generate superoxide radicals (O₂⁻) via the reaction: 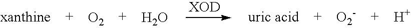
Solutions:
Cytochrome C stock:
17.3 mg cytochrome C (from horse heart)
2 mL distilled, deionized water
   Reagent Cocktail (“RC”):
800 uL cytochrome c stock
9.2 mL PBS (1 mM EDTA)
   Xanthine stock:
0.0076 g xanthine
1 mL 1.5 M NaOH
19 mL distilled, deionized water
   Xanthine working solution:
4.2 mL xanthine stock
14.77 mL distilled, deionized water
1.025 mL 1.2M HCl
105 uL 1.5M NaOH
→Measured pH (with pH meter)=7.46
→[X]=0.525 mM
   XOD solution:
40 uL XOD suspension (18 mg prot/mL, 0.6 U/mg)
2 mL distilled, deionized water (cold)
→[XOD]=0.22 U/mL
   2X-PBS: 2 PBS packets from Aldrich
1 L distilled water
0.0195 g disodium EDTA
0.6245 g trisodium EDTA
   Gel preparation:

Slabs with and without SODm were polymerized according to the procedure described above. In this experiment, slabs containing 1 and 5% SODm were used. Three or four 1 cm² 100 um pieces of each slab gel type were ground (using a standard tissue grinder) into a fine suspension and suspended in 1 mL PBS only (no EDTA) in 1.5 mL microcentrifuge tubes.

Assay procedure:

1. Prepare oxidant solution immediately prior to use:
   Oxidant solution:
      250 uL xanthine working solution
      775 uL 2X-PBS
      25 uL XOD solution
2. Set up three test tubes:
   1) Cytochrome C only (negative control):
      1 mL PBS (no EDTA)
      300 uL RC solution
      50 uL PBS (with 1 mM EDTA)
   2) Solution reaction (positive control)
      1 mL PBS (no EDTA)
      300 uL RC solution
   3) Ground gel.
      1 mL PBS (no EDTA)
      300 uL RC solution
3. Starting with ground gel (tube 3), vortex to mix, then add 50 uL oxidant solution to tube 3, followed by tube 2.
4. Mix all three tubes by inversion continuously for 5 minutes.
5. After 5 minutes, centrifuge all 3 samples 6 minutes. Record the total time of reaction (mixing+centrifuging).
6. Run absorbance spectra on the supernatant from each tube using a 1 cm pathlength quartz cuvette.
7. Monitor absorbance at:
   a=540 nm, b=560 nm, c=550 nm
8. Calculate Absolute Magnitude of 550 nm peak as follows:
   Abs. Magn.=c−[(a+b)/2]
9. Calculate % inhibition of cytochrome c in gel as follows:
   % inhibition=100−[(Abs. Magn. tube 3/Abs. Magn. tube 2)×100]
      Results:

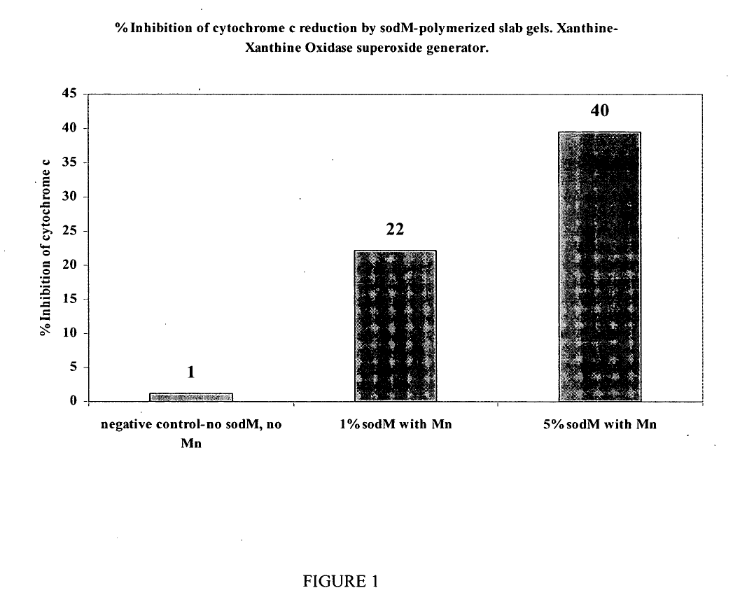

The data in FIG. 1 show that the inhibition of cytochrome C reduction is greatly increased by the copolymerized inclusion of the SODm monomer into the slab material.

We claim:

1. An implantable device for detecting the presence or concentration of an analyte in an aqueous environment in vivo, said device including a macromolecule that comprises a copolymer of:
a) one or more indicator component monomers which individually are not sufficiently water soluble to permit their use in an aqueous environment for detecting the presence or concentration of said analyte;
b) one or more hydrophilic monomers; and
c) one or more catalytic antioxidant monomers;
such that the macromolecule is capable of detecting the presence or concentration of said analyte in an aqueous environment.

2. The implantable device of claim 1, wherein the macromolecule is capable of detection by an optical change.

3. The implantable device of claim 1, wherein the indicator component monomer comprises an N-(o-boronobenzyl)aminomethylanthracene derivative.

4. The implantable device of claim 3, wherein the indicator component monomer is selected from the group consisting of
9-[[N-methacryloylaminopropyl-N-(o-boronobenzyl)amino]-methyl]anthracene;
9-[N-[2-(5,5-dimethyl-[1,3,2]dioxaborinan-2-yl)benzyl]-N-[3-(methacrylamido)propylamino]methyl]-10-[N-[2-(5,5-dimethyl-[1,3,2]dioxaborinan-2-yl)benzyl]-N-[2-(2-hydroxyethoxy)-ethylamino]methyl]anthracene;
9-[N-(2-boronobenzyl)-N-[3-(methacrylamido)-propylamino]methyl]-10-[N-(2-boronobenzyl)-N-[2-(2-hydroxyethoxy)ethylamino]methyl]anthracene;
9,10-bis[N-[2-(5,5-dimethyl-[1,3,2]dioxaborinan-2-yl)benzyl]-N-[3-(methacrylamido)propylamino]methyl]anthracene;
9,10-bis[N-(2-boronobenzyl)-N-[3-(methacrylamido)-propylamino]methyl]anthracene;
9-[N-[2-(5,5-dimethyl-[1,3,2]dioxaborinan-2-yl)benzyl]-N-[2-(2-methacroyloxyethoxy)ethylamino]methyl]-10-[N-[2-(5,5-dimethyl-[1,3,2]dioxaborinan-2-yl)benzyl]-N-[2-(2-hydroxyethoxy)-ethylamino]methyl]anthracene;
9-[N-(2-boronobenzyl)-N-[2-(2-methacroyloxyethoxy)-ethylamino]methyl]-10-[N-[2-boronobenzyl)]-N-[2-(2-hydroxyethoxy)ethylamino]methyl]anthracene;
9,10-bis[N-[2-(5,5-dimethyl-[1,3,2]dioxaborinan-2-yl)benzyl]-N-[2-(2-methacroyloxyethoxy)ethylamino]methyl]anthracene;
9,10-bis[N-(2-boronobenzyl)-N-[2-(2-methacroyloxyethoxy)ethylamino]methyl]anthracene;
9-[N-(2-boronobenzyl)-N-[3-(methacrylamido)propylamino]methyl]-10-[N-(2-boronobenzyl)-N-[2-(carboxyethyl)amino]methyl]anthracene;
9-[N-(2-boronobenzyl)-N-[3-(methacrylamido)propylamino]methyl]-10-[N-(2-boronobenzyl)-N-[6-(cyclohexanecarboxamido)hexylamino]methyl]anthracene;
9-[N-[2-(4,4,5,5-tetramethyl-1,3,2-dioxaborolano)benzyl]-N-[3-(methacrylamido)propylamino]methyl]-10-[N—[2-(4,4,5,5-tetramethyl-1,3,2-dioxaborolano)benzyl]-N-[2-(carboxyethyl)amino]methyl]anthracene;
and salts or derivatives thereof.

5. The implantable device of claim 3, wherein the hydrophilic monomer comprises [3-(methacryloylamino)-propyl]trimethylammonium chloride.

6. The implantable device of claim 1, wherein the indicator component monomer is selected from the group consisting of a lanthanide chelate and a polyaromatic hydrocarbon.

7. The implantable device of claim 1, wherein the molar ratio of hydrophilic monomer:indicator component momomer is from about 2:1 to about 1000:1.

8. The implantable device of claim 7, wherein the ratio of hydrophilic monomer:indicator component momomer is from about 5:1 to about 50:1.

9. The implantable device of claim 8, wherein the ratio of hydrophilic monomer:indicator component momomer is about 5:1.

10. The implantable device of claim 1, wherein the analyte detected is selected from the group consisting of a vicinal diol; an α-hydroxy acid, a β-keto acid oxygen; carbon dioxide; zinc, potassium, hydrogen, or carbonate ions; a toxin; a mineral; and a hormone.

11. The implantable device of claim 10, wherein the analyte detected is a vicinal diol which comprises a saccharide.

12. The implantable device of claim 11, wherein the saccharide is glucose.

13. The implantable device of claim 1, wherein
i) the molar ratio of hydrophilic monomer:indicator component momomer is from about 2:1 to about 15:1,
ii) the indicator component monomer comprises an N-(o-boronobenzyl)amino]methyl]anthracene derivative,
iii) the hydrophilic monomer comprises [3-(methacryloylamino)propyl]trimethylammonium chloride, and
iv) the macromolecule exhibits an excimer effect.

14. The implantable device of claim 1, wherein the hydrophilicity of the hydrophilic monomer is increased by the addition of a functional constituent.

15. The implantable device of claim 14, wherein the functional constituent is selected from the group consisting of an ion, a polar moiety, a halogen, and combinations thereof.

16. The implantable device of claim 15, wherein the ion is selected from the group consisting of sulfonate, quartenary amine, carboxyl, and combinations thereof.

17. The implantable device of claim 15, wherein the polar moiety is selected from the group consisting of hydroxyl, sulfhydryl, an amine, carbonyl, an amide, and combinations thereof.

18. The implantable device of claim 1, wherein the catalytic antioxidant monomer comprises a superoxide dismutase mimic monomer which is selected from the group consisting of derivatives of:
CAS RN 218791-21-0;
CAS RN 218791-27-6;
CAS RN 218791-28-7;
CAS RN 301664-32-4;
CAS RN 301664-47-1;
CAS RN 311767-56-3;
CAS RN 311767-57-4;
CAS RN 311767-59-6;
CAS RN 330626-36-3;
CAS RN 330626-39-6;
CAS RN 330626-40-9;
CAS RN 330626-41-0;
CAS RN 330626-42-1;
CAS RN 330626-43-2;
CAS RN 330626-45-4;
CAS RN 330626-47-6;
CAS RN 330626-49-8;
CAS RN 330626-51-2;
CAS RN 330626-52-3;
CAS RN 330626-53-4;
CAS RN 330626-54-5;
CAS RN 330626-55-6;
CAS RN 330626-56-7;
CAS RN 330626-57-8;
CAS RN 330626-58-9;
CAS RN 330626-59-0;
CAS RN 330626-60-3;
CAS RN 330626-61-4;
CAS RN 330626-62-5;
CAS RN 330626-63-6;
CAS RN 330626-64-7;
CAS RN 330626-65-8;
CAS RN 330626-66-9;
CAS RN 330626-67-0;
CAS RN 330626-68-1;
CAS RN 330626-69-2;
CAS RN 330626-70-5;
CAS RN 330626-71-6;
CAS RN 330626-72-7;
CAS RN 330626-73-8;
CAS RN 330626-74-9;
CAS RN 330626-75-0;
CAS RN 330626-76-1;
CAS RN 331718-71-9; and
CAS RN 331718-72-0.

19. A method for detecting the presence or concentration of an analyte in a sample having an aqueous environment in vivo, said method comprising:
a) exposing the sample to an implantable device that includes an indicator macromolecule, said macromolecule comprising a copolymer of:
i) one or more indicator component monomers which individually are not sufficiently water soluble to permit their use in an aqueous environment for detecting the presence or concentration of said analyte;
ii) one or more hydrophilic monomers; and
iii) one or more catalytic antioxidant monomers;
such that the resulting macromolecule is capable of detecting the presence or concentration of said analyte in an aqueous environment, and wherein the indicator macromolecule has a detectable quality that changes in a concentration-dependent manner when said macromolecule is exposed to said analyte; and
b) measuring any change in said detectable quality to thereby determine the presence or concentration of said analyte in said sample.

20. The method of claim 19, wherein the change in said detectable quality is an optical change.

21. The method of claim 19, wherein the indicator component monomer comprises an N-(o-boronobenzyl)aminomethylanthracene derivative.

22. The method of claim 21, wherein the indicator component monomer is selected from the group consisting of
9-[[N-methacryloylaminopropyl-N-(o-boronobenzyl)amino]-methyl]anthracene;
9-[N-[2-(5,5-dimethyl-[1,3,2]dioxaborinan-2-yl)benzyl]-N-[3-(methacrylamido)propylamino]methyl]-10-[N-[2-(5,5-dimethyl-[1,3,2]dioxaborinan-2-yl)benzyl]-N-[2-(2-hydroxyethoxy)-ethylamino]methyl]anthracene;
9-[N-(2-boronobenzyl)-N-[3-(methacrylamido)-propylamino]methyl]-10-[N-(2-boronobenzyl)-N-[2-(2-hydroxyethoxy)ethylamino]methyl]anthracene;
9,10-bis[N-[2-(5,5-dimethyl-[1,3,2]dioxaborinan-2-yl)benzyl]-N-[3-(methacrylamido)propylamino]methyl]anthracene;
9,10-bis[N-(2-boronobenzyl)-N-[3-(methacrylamido)-propylamino]methylanthracene;
9-[N-[2-(5,5-dimethyl-[1,3,2]dioxaborinan-2-yl)benzyl]-N-[2-(2-methacroyloxyethoxy)ethylamino]methyl]-10-[N-[2-(5,5-dimethyl-[1,3,2]dioxaborinan-2-yl)benzyl]-N-[2-(2-hydroxyethoxy)-ethylamino]methyl]anthracene;
9-[N-(2-boronobenzyl)-N-[2-(2-methacroyloxyethoxy)-ethylamino]methyl]-10-[N-[2-boronobenzyl)]-N-[2-(2-hydroxyethoxy)ethylamino]methyl]anthracene;
9,10-bis[N-[2-(5,5-dimethyl-[1,3,2]dioxaborinan-2-yl)benzyl]-N-[2-(2-methacroyloxyethoxy)ethylamino]methyl]anthracene;
9,10-bis[N-(2-boronobenzyl)-N-[2-(2-methacroyloxyethoxy)ethylamino]methyl]anthracene;
9-[N-(2-boronobenzyl)-N-[3-(methacrylamido)propylamino]methyl]-10-[N-(2-boronobenzyl)-N-[2-(carboxyethyl)amino]methyl]anthracene;
9-[N-(2-boronobenzyl)-N-[3-(methacrylamido)propylamino]methyl]-10-[N-(2-boronobenzyl)-N-[6-(cyclohexanecarboxamido)hexylamino]methyl]anthracene;
9-[N-[2-(4,4,5,5-tetramethyl-1,3,2-dioxaborolano)benzyl]-N-[3-(methacrylamido)propylamino]methyl]-10-[N—[2-(4,4,5,5-tetramethyl-1,3,2-dioxaborolano)benzyl]-N-[2-(carboxyethyl)amino]methyl]anthracene;
and salts or derivatives thereof.

23. The method of claim 19, wherein the hydrophilic monomer comprises [3-(methacryloylamino)-propyl]trimethylammonium chloride.

24. The method of claim 19, wherein the indicator component monomer is selected from the group consisting of a lanthanide chelate and a polyaromatic hydrocarbon.

25. The method of claim 19, wherein the molar ratio of hydrophilic monomer:indicator component momomer is from about 2:1 to about 1000:1.

26. The method of claim 25, wherein the ratio of hydrophilic monomer:indicator component momomer is from about 5:1 to about 50:1.

27. The method of claim 26, wherein the ratio of hydrophilic monomer:indicator component momomer is about 5:1.

28. The method of claim 19, wherein the analyte detected is selected from the group consisting of a vicinal diol; an α-hydroxy acid; a β-keto acid; oxygen; carbon dioxide; zinc, potassium, hydrogen, or carbonate ions; a toxin; a mineral; and a hormone.

29. The method of claim 28, wherein the analyte detected is a vicinal diol which comprises a saccharide.

30. The method of claim 29, wherein the saccharide is glucose.

31. The method of claim 19, wherein
i) the molar ratio of hydrophilic monomer:indicator component momomer is from about 2:1 to about 15:1,
ii) the indicator component monomer comprises an N-(o-boronobenzyl)aminomethylanthracene derivative,
iii) the hydrophilic monomer comprises [3-(methacryloylamino)propyl]trimethylammonium chloride, and
iv) the macromolecule exhibits an excimer effect.

32. The method of claim 31, wherein said macromolecule serves as both an indicator and a reference.

33. The method of claim 19, wherein the hydrophilicity of the hydrophilic monomer is increased by the addition of a functional constituent.

34. The method of claim 33, wherein the functional constituent is selected from the group consisting of an ion, a polar moiety, a halogen, and combinations thereof.

35. The method of claim 34, wherein the ion is selected from the group consisting of sulfonate, quartenary amine, carboxyl, and combinations thereof.

36. The method of claim 34, wherein the polar moiety is selected from the group consisting of hydroxyl, sulfhydryl, an amine, carbonyl, an amide, and combinations thereof.

37. The method of claim 19, wherein the catalytic antioxidant monomer comprises a superoxide dismutase mimic monomer which is selected from the group consisting of derivatives of:
CAS RN 218791-21-0;
CAS RN 218791-27-6;
CAS RN 218791-28-7;
CAS RN 301664-32-4;
CAS RN 301664-47-1;
CAS RN 311767-56-3;
CAS RN 311767-57-4;
CAS RN 311767-59-6;
CAS RN 330626-36-3;
CAS RN 330626-39-6;
CAS RN 330626-40-9;
CAS RN 330626-41-0;
CAS RN 330626-42-1;
CAS RN 330626-43-2;
CAS RN 330626-45-4;
CAS RN 330626-47-6;
CAS RN 330626-49-8;
CAS RN 330626-51-2;
CAS RN 330626-52-3;
CAS RN 330626-53-4;
CAS RN 330626-54-5;
CAS RN 330626-55-6;
CAS RN 330626-56-7;
CAS RN 330626-57-8;
CAS RN 330626-58-9;
CAS RN 330626-59-0;
CAS RN 330626-60-3;
CAS RN 330626-61-4;
CAS RN 330626-62-5;
CAS RN 330626-63-6;
CAS RN 330626-64-7;
CAS RN 330626-65-8;
CAS RN 330626-66-9;
CAS RN 330626-67-0;
CAS RN 330626-68-1;
CAS RN 330626-69-2;
CAS RN 330626-70-5;
CAS RN 330626-71-6;
CAS RN 330626-72-7;
CAS RN 330626-73-8;
CAS RN 330626-74-9;
CAS RN 330626-75-0;
CAS RN 330626-76-1;
CAS RN 331718-71-9; and
CAS RN 331718-72-0.

38. An implantable device that is capable of exhibiting an excimer effect, said device including a macromolecule which comprises a copolymer of:
a) one or more excimer forming monomers, the molecules of which are capable of exhibiting an excimer effect when suitably oriented with respect to each other; and
b) one or more other monomers comprising a catalytic antioxidant;
such that the resulting macromolecule exhibits said excimer effect.

39. The implantable device of claim 38, wherein the macromolecule is capable of detecting the presence or concentration of an analyte.

40. The implantable device of claim 39, wherein
a) the excimer forming monomer individually is not sufficiently water soluble to permit its use in an aqueous environment for detecting the presence or concentration of said analyte; and
b) the other monomer is a hydrophilic monomer;
such that the macromolecule is capable of detecting the presence or concentration of said analyte in an aqueous environment.

41. The implantable device of claim 39, wherein the excimer effect does not substantially change in response to changes in the presence or concentration of the analyte.

42. The implantable device of claim 41, wherein
i) the molar ratio of other monomer:excimer forming momomer is from about 2:1 to about 15:1,
ii) the excimer forming monomer comprises an N-(o-boronobenzyl)aminomethylanthracene derivative, and
iii) the other monomer comprises [3-(methacryloylamino)propyl]trimethylammonium chloride.

43. The implantable device of claim 38, wherein the excimer forming monomer is selected from the group consisting of a lanthanide chelate and a polyaromatic hydrocarbon.

44. The implantable device of claim 42, wherein the excimer forming monomer is selected from the group consisting of
9-[[N-methacryloylaminopropyl-N-(o-boronobenzyl)amino]-methyl]anthracene;
9-[N-[2-(5,5-dimethyl-[1,3,2]dioxaborinan-2-yl)benzyl]-N-[3-(methacrylamido)propylamino]methyl]-10-[N-[2-(5,5-dimethyl-[1,3,2]dioxaborinan-2-yl)benzyl]-N-[2-(2-hydroxyethoxy)-ethylamino]methyl]anthracene;
9-[N-(2-boronobenzyl)-N-[3-(methacrylamido)-propylamino]methyl]-10-[N-(2-boronbenzyl)-N-[2-(2-hydroxyethoxy)ethylamino]methyl]anthracene;
9,10-bis[N-[2-(5,5-dimethyl-[1,3,2]dioxaborinan-2-yl)benzyl]-N-[3-(methacrylamido)propylamino]methyl]anthracene;
9,10-bis[N-(2-boronobenzyl)-N-[3-(methacrylamido)-propylamino]methylanthracene;
9-[N-[2-(5,5-dimethyl-[1,3,2]dioxaborinan-2-yl)benzyl]-N-[2-(2-methacroyloxyethoxy)ethylamino]methyl]-10-[N-[2-(5,5-dimethyl-[1,3,2]dioxaborinan-2-yl)benzyl]-N-[2-(2-hydroxyethoxy)-ethylamino]methyl]anthracene;
9-[N-(2-boronobenzyl)-N-[2-(2-methacroyloxyethoxy)-ethylamino]methyl]-10-[N-(2-boronobenzyl)-N-[2-(2-hydroxyethoxy)ethylamino]methyl]anthracene;
9,10-bis[N-[2-(5,5-dimethyl-[1,3,2]dioxaborinan-2-yl)benzyl]-N-[2-(2-methacroyloxyethoxy)ethylamino]methyl]anthracene;
9,10-bis[N-(2-boronobenzyl)-N-[2-(2-methacroyloxyethoxy)ethylamino]methyl]anthracene;
9-[N-(2-boronobenzyl)-N-[3-(methacrylamido)propylamino]methyl]-10-[N-(2-boronobenzyl)-N-[2-(carboxyetbyl)amino]methyl]anthracene;
9-[N-(2-boronobenzyl)-N-[3-(methacrylamido)propylamino]methyl]-10-[N-(2-boronobenzyl)-N-[6-(cyclohexanecarboxamido)hexylamino]methyl]anthracene;
9-[N-[2-(4,4,5,5-tetramethyl-1,3,2-dioxaborolano)benzyl]-N-[3-(methacrylamido)propylamino]methyl]-10-[N—[2-(4,4,5,5-tetramethyl-1,3,2-dioxaborolano)benzyl]-N-[2-(carboxyethyl)amino]methyl]anthracene;
and salts or derivatives thereof.

45. The implantable device of claim 38, wherein the hydrophilicity of the hydrophilic monomer is increased by the addition of a functional constituent.

46. The implantable device of claim 45, wherein the functional constituent is selected from the group consisting of an ion, a polar moiety, a halogen, and combinations thereof.

47. The implantable device of claim 46, wherein the ion is selected from the group consisting of sulfonate, quartenary amine, carboxyl, and combinations thereof.

48. The implantable device of claim 46, wherein the polar moiety is selected from the group consisting of hydroxyl, sulfhydryl, an amine, carbonyl, an amide, and combinations thereof.

49. The implantable device of claim 38, wherein the catalytic antioxidant monomer comprises a superoxide dismutase mimic monomer which is selected from the group consisting of derivatives of:
CAS RN 218791-21-0;
CAS RN 218791-27-6;
CAS RN 218791-28-7;
CAS RN 301664-32-4;
CAS RN 301664-47-1;
CAS RN 311767-56-3;
CAS RN 311767-57-4;
CAS RN 311767-59-6;
CAS RN 330626-36-3;
CAS RN 330626-39-6;
CAS RN 330626-40-9;
CAS RN 330626-41-0;
CAS RN 330626-42-1;
CAS RN 330626-43-2;
CAS RN 330626-45-4;
CAS RN 330626-47-6;
CAS RN 330626-49-8;
CAS RN 330626-51-2;
CAS RN 330626-52-3;
CAS RN 330626-53-4;
CAS RN 330626-54-5;
CAS RN 330626-55-6;
CAS RN 330626-56-7;
CAS RN 330626-57-8;
CAS RN 330626-58-9;
CAS RN 330626-59-0;
CAS RN 330626-60-3;
CAS RN 330626-61-4;
CAS RN 330626-62-5;
CAS RN 330626-63-6;
CAS RN 330626-64-7;
CAS RN 330626-65-8;
CAS RN 330626-66-9;
CAS RN 330626-67-0;
CAS RN 330626-68-1;
CAS RN 330626-69-2;
CAS RN 330626-70-5;
CAS RN 330626-71-6;
CAS RN 330626-72-7;
CAS RN 330626-73-8;
CAS RN 330626-74-9;
CAS RN 330626-75-0;
CAS RN 330626-76-1;
CAS RN 331718-71-9; and
CAS RN 331718-72-0.

50. A method for detecting the presence or concentration of an analyte in a sample in vivo, said method comprising:
a) exposing the sample to an an implantable device that includes an indicator macromolecule, said macromolecule comprising a copolymer of:
i) one or more indicator component monomers, the molecules of which are capable of exhibiting an excimer effect when suitably oriented with respect to each other, and which are also capable of detecting the presence or concentration of an analyte; and
ii) one or more other monomers, comprising a catalytic antioxidant;
such that the resulting macromolecule exhibits said excimer effect, and wherein the indicator macromolecule has a detectable quality that changes in a concentration-dependent manner when said macromolecule is exposed to said analyte; and
b) measuring any change in said detectable quality to thereby determine the presence or concentration of said analyte in said sample.

51. The method of claim 50, wherein the excimer effect does not substantially change in response to changes in the presence or concentration of the analyte.

52. The method of claim 51, wherein
i) the molar ratio of other monomer:indicator component momomer is from about 2:1 to about 15:1,
ii) the indicator component monomer comprises an N-(o-boronobenzyl)amino]methyl]anthracene derivative, and
iii) the other monomer comprises [3-(methacryloylamino)propyl]trimethylammonium chloride.

53. The method of claim 50, wherein the indicator component monomer is selected from the group consisting of a lanthanide chelate and a polyaromatic hydrocarbon.

54. The method of claim 51, wherein the indicator component monomer is selected from the group consisting of
9-[[N-methacryloylaminopropyl-N-(o-boronobenzyl)amino]-methyl]anthracene;
9-[N-(2-boronobenzyl)-N-[2-(2-methacroyloxyethoxy)-ethylamino]methyl]-10-[N-(2-boronobenzyl)-N-[2-(2-hydroxyethoxy)ethylamino]methyl]-anthracene; and
9,10-bis[N-(2-boronobenzyl)-N-[2-(2-methacroyloxyethoxy)ethylamino]methyl]anthracene;
9-[N-[2-(5,5-dimethyl-[1,3,2]dioxaborinan-2-yl)benzyl]-N-[3-(methacrylamido)propylamino]methyl]-10-[N-[2-(5,5-dimethyl-[1,3,2]dioxaborinan-2-yl)benzyl]-N-[2-(2-hydroxyethoxy)-ethylamino]methyl]anthracene;
9-[N-(2-boronobenzyl)-N-[3-(methacrylamido)-propylamino]methyl]-10-[N-(2-boronobenzyl)-N-[2-(2-hydroxyethoxy)ethylamino]methyl]anthracene;
9,10-bis[N-[2-(5,5-dimethyl-[1,3,2]dioxaborinan-2-yl)benzyl]-N-[3-(methacrylamido)propylamino]methyl]anthracene;
9,10-bis[N-(2-boronobenzyl)-N-[3-(methacrylamido)-propylamino]methyl]anthracene;
9-[N-[2-(5,5-dimethyl-[1,3,2]dioxaborinan-2-yl)benzyl]-N-[2-(2-methacroyloxyethoxy)ethylamino]methyl]-10-[N-[2-(5,5-dimethyl-[1,3,2]dioxaborinan-2-yl)benzyl]-N-[2-(2-hydroxyethoxy)-ethylamino]methyl]anthracene;
9,10-bis[N-[2-(5,5-dimethyl-[1,3,2]dioxaborinan-2-yl)benzyl]-N-[2-(2-methacroyloxyethoxy)ethylamino]methyl]anthracene;
N-[3-(methacrylamido)propyl]-3,4-dihydroxy-9,10-dioxo-2-anthracenesulfonamide;
α,α′-bis[N-[2-(5,5-dimethyl-[1,3,2]dioxaborinan-2-yl)benzyl]-N-[3-(methacrylamido)propylamino]-1,4-xylene;
9-[N-(2-boronobenzyl)-N-[3-(methacrylamido)propylamino]methyl]-10-[N-(2-boronobenzyl)-N-[2-(carboxyethyl)amino]methyl]anthracene;
9-[N-(2-boronobenzyl)-N-[3-(methacrylamido)propylamino]methyl]-10-[N-(2-boronobenzyl)-N-[6-(cyclohexanecarboxamido)hexylamino]methyl]anthracene;
9-[N-[2-(4,4,5,5-tetramethyl-1,3,2-dioxaborolano)benzyl]-N-[3-(methacrylamido)propylamino]methyl]-10-[N—[2-(4,4,5,5-tetramethyl-1,3,2-dioxaborolano)benzyl]-N-[2-(carboxyethyl)amino]methyl]anthracene;
and salts or derivatives thereof.

55. The method of claim 50, wherein the hydrophilicity of the hydrophilic monomer is increased by the addition of a functional constituent.

56. The method of claim 55, wherein the functional constituent is selected from the group consisting of an ion, a polar moiety, a halogen, and combinations thereof.

57. The method of claim 56, wherein the ion is selected from the group consisting of sulfonate, quartenary amine, carboxyl, and combinations thereof.

58. The method of claim 56, wherein the polar moiety is selected from the group consisting of hydroxyl, sulfhydryl, an amine, carbonyl, an amide, and combinations thereof.

59. The implantable device of claim 1, wherein the indicator component monomer is selected from the group consisting of:
N-[3-(methacrylamido)propyl]-3,4-dihydroxy-9,10-dioxo-2-anthracenesulfonamide;
α,α′-bis[N-[2-(5,5-dimethyl-[1,3,2]dioxaborinan-2-yl)benzyl]-N-[3-(methacrylamido)propylamino]-1,4-xylene;
and salts or derivatives thereof.

60. The method of claim 19, wherein the indicator component monomer is selected from the group consisting of:
N-[3-(methacrylamido)propyl]-3,4-dihydroxy-9,10-dioxo-2-anthracenesulfonamide;
α,α′-bis[N-[2-(5,5-dimethyl-[1,3,2]dioxaborinan-2-yl)benzyl]-N-[3-(methacrylamido)propylamino]-1,4-xylene;
and salts or derivatives thereof.

61. The implantable device of claim 38, wherein the excimer forming monomer is selected from the group consisting of:
N-[3-(methacrylamido)propyl]-3,4-dihydroxy-9,10-dioxo-2-anthracenesulfonamide;
α,α′-bis[N-[2-(5,5-dimethyl-[1,3,2]dioxaborinan-2-yl)benzyl]-N-[3-(methacrylamido)propylamino]-1,4-xylene;
and salts or derivatives thereof.

62. The method of claim 50, wherein the indicator component monomer is selected from the group consisting of:
N-[3-(methacrylamido)propyl]-3,4-dihydroxy-9,10-dioxo-2-anthracenesulfonamide;
α,α′-bis[N-[2-(5,5-dimethyl-[1,3,2]dioxaborinan-2-yl)benzyl]-N-[3-(methacrylamido)propylamino]-1,4-xylene;
and salts or derivatives thereof.

63. An implantable device for detecting the presence or concentration of an analyte in an aqueous environment in vivo, said device including a macromolecule that comprises a copolymer of:
a) one or more indicator component monomers which individually are not sufficiently water soluble to permit their use in an aqueous environment for detecting the presence or concentration of said analyte; and
b) one or more hydrophilic monomers;
such that the macromolecule is capable of detecting the presence or concentration of said analyte in an aqueous environment, and wherein at least one catalytic antioxidant is attached to said macromolecule.

64. The implantable device of claim 63, wherein the catalytic antioxidant is covalently attached to the macromolecule.

65. The implantable device of claim 63, wherein the catalytic antioxidant comprises a superoxide dismutase mimic.

66. The implantable device of claim 65, wherein the superoxide dismutase mimic is selected from the group consisting of derivatives of:
CAS RN 218791-21-0;
CAS RN 218791-27-6;
CAS RN 218791-28-7;
CAS RN 301664-32-4;
CAS RN 301664-47-1;
CAS RN 311767-56-3;
CAS RN 311767-57-4;
CAS RN 311767-59-6;
CAS RN 330626-36-3;
CAS RN 330626-39-6;
CAS RN 330626-40-9;
CAS RN 330626-41-0;
CAS RN 330626-42-1;
CAS RN 330626-43-2;
CAS RN 330626-45-4;
CAS RN 330626-47-6;
CAS RN 330626-49-8;
CAS RN 330626-51-2;
CAS RN 330626-52-3;
CAS RN 330626-53-4;
CAS RN 330626-54-5;
CAS RN 330626-55-6;
CAS RN 330626-56-7;
CAS RN 330626-57-8;
CAS RN 330626-58-9;
CAS RN 330626-59-0;
CAS RN 330626-60-3;
CAS RN 330626-61-4;
CAS RN 330626-62-5;
CAS RN 330626-63-6;
CAS RN 330626-64-7;
CAS RN 330626-65-8;
CAS RN 330626-66-9;
CAS RN 330626-67-0;
CAS RN 330626-68-1;
CAS RN 330626-69-2;
CAS RN 330626-70-5;
CAS RN 330626-71-6;
CAS RN 330626-72-7;
CAS RN 330626-73-8;
CAS RN 330626-74-9;
CAS RN 330626-75-0;
CAS RN 330626-76-1;
CAS RN 331718-71-9; and
CAS RN 331718-72-0.
